(19) United States
(12) Patent Application Publication
(10) Pub. No.: US 2024/0353393 A1
(43) Pub. Date: Oct. 24, 2024

(54) LABEL FREE DRUG AND TISSUE BIOPSY SCREENING/DIAGNOSTIC IN CANCER

(71) Applicant: University of Virginia Patent Foundation, Charlottesville, VA (US)

(72) Inventors: Ammasi Periasamy, Crozet, VA (US); Shagufta R. Alam, Charlottesville, VA (US)

(21) Appl. No.: 18/645,041

(22) Filed: Apr. 24, 2024

Related U.S. Application Data
(60) Provisional application No. 63/497,814, filed on Apr. 24, 2023.

(51) Int. Cl. G01N 33/50; G01N 21/64; G16H 50/20

(52) U.S. Cl. CPC G01N 33/5011; G01N 21/6486; G01N 33/5014; G16H 50/20; G01N 2800/7028

(57) ABSTRACT

Provided herein is a drug screening method as well as an automatic screening of biopsy tissue to identify and grade cancer progression without any labeling.

### PRIORITY

This application claims the benefit of priority of U.S. Provisional Patent Application No. 63/497,814, filed on Apr. 24, 2023, the benefit of priority of which is claimed hereby, and which is incorporated by reference herein in its entirety.

### BACKGROUND

Prostate cancer (PCa) is a leading cause of cancer and the second leading cause of death in men in the USA (CDC, USA). Alarmingly, the incidence of PCa has been on the rise and 1 in 7 men will be diagnosed with PCa in their lifetime. Increasingly, men with PCa are diagnosed late at advanced stages, where 35% of the cases have recurrence leading to transformation into a castration resistant prostate cancer (CRPC) phenotype, the most deadly and aggressive form of PCa where standard chemotherapies are largely ineffective. Early diagnosis, where chemotherapeutics is most effective will help ameliorate the burden of PCa and other cancers.

### SUMMARY

Provided herein is a label free drug screening, diagnostic method, and device in cancer, including tissue biopsies and cells.

One embodiment provides a method to screen for cancer treatment compounds comprising contacting a cancer cell with a test compound and determining the ratio of bound NAD (P) H to bound FAD (the fluorescence lifetime imaging redox ratio (FLIRR)) via fluorescence lifetime imaging microscopy (FLIM), wherein an increased ratio of NAD (P) H to FAD, as compared to a cancer cell that has not been contacted with the test compound, indicates a cancer treatment compound. In one embodiment, the compounds are further tested for cytotoxicity and heterogeneity in drug response using co-cultures of the cancer and non-cancerous cells. In one embodiment, wherein the cells are fibroblasts. One embodiment further comprises determining Trp-NAD (P) H FRET interactions in the cancer cells, wherein the Trp-NAD (P) H FRET interactions are determined in a 3D model spheroid of cancer cells. In one embodiment the FLIM is multiphoton FLIM. In one embodiment photomultiplier tube (PMT) based metabolic imaging or SPAD camera with FLIM is used. In one embodiment, FLIRR is calculated at each pixel of the image. In one embodiment, the calculation uses time correlated single photon counting or fast FLIM frequency domain. In one embodiment, the FLIM analysis is carried out by maximum likelihood (MLE) fitting methods or phasor analysis.

One embodiment provides a method to diagnose cancer a tissue biopsy comprising determining the ratio of bound NAD (P) H to bound FAD (the fluorescence lifetime imaging redox ratio (FLIRR)) via fluorescence lifetime imaging microscopy (FLIM) in said biopsy, wherein a decreased ratio of NAD (P) H to FAD, as compared to a non-cancerous biopsy indicates a cancer. In one embodiment, the FLIM is multiphoton FLIM. In one embodiment, photomultiplier tube (PMT) based metabolic imaging or SPAD camera with FLIM is used. In one embodiment, FLIRR is calculated at each pixel of the image. In one embodiment, the calculation uses time correlated single photon counting or fast FLIM frequency domain. In one embodiment, the FLIM analysis is carried out by maximum likelihood (MLE) fitting methods or phasor analysis. In one embodiment, the biopsy is fresh or frozen and/or thawed. In one embodiment, the cancer is a solid tumor such as a sarcoma or carcinoma. In one embodiment, the cancer is a fibrosarcoma, myxosarcoma, liposarcoma, chondrosarcoma, osteogenic sarcoma, osteosarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, pancreatic cancer, pancreatic ductal adenocarcinoma, breast cancer, ovarian cancer, prostate cancer, squamous cell carcinoma, basal cell carcinoma, adenocarcinoma, sweat gland carcinoma, sebaceous gland carcinoma, carcinoma, papillary papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilm's tumor, cervical cancer, uterine cancer, testicular cancer, lung carcinoma, small cell lung carcinoma, bladder carcinoma, epithelial carcinoma, glioma, astrocytoma, medulloblastoma, craniopharyngioma, ependymoma, pincaloma, hemangioblastoma, acoustic neuroma, oligodenroglioma, schwannoma, meningioma, melanoma, neuroblastoma, and retinoblastoma. In one embodiment, the cancer is prostate cancer. In one embodiment, no exogenous label or dye is used. In one embodiment, the data is used by artificial intelligence (AI) to generate a data display and/or for cancer evaluation. One embodiment further comprises grading the cancer. One embodiment further comprises treating the diagnosed cancer.

### BRIEF DESCRIPTION OF THE DRAWINGS

FIG. 1. Neoplastic transformation in prostate epithelium. Quantitative ML-based FLIRR for early prostate cancer detection vs qualitative H&E based Gleason grading.

FIGS. 2A-2B. FLIRR mosaic of 256 images (16×16) tiled at 128×128 pixel size of tissue section from the same PCa patient. (A) normal region and (B) PCa tumor confirmed by H&E staining with the Gleason score of 7 and 9.21 blood PSA level. Even though (A) was diagnosed as normal by H&E it shows, some regions of lower FLIRR values in the color coded FLIRR mosaic which is indicative of defective OXPHOS and growing tumor. As molecular changes precede phenotypic or morphological changes (4,43), the sensitivity of FLIRR outperforms H&E (FIG. 1).

FIG. 3. In one embodiment, Grading can be as follows: FLIRR grading system of a tumor was based on FLIRR score and compared with H&E staining and Gleason scoring. Top panel-H&E-stained images; Bottom panel-FLIRR images.

FIG. 4. FLIRR mosaic of 256 images (16×16) tiled at 128×128 pixel size for Prostate cancer biopsy flash frozen tissue section by simultaneous excitation using 2p-800 nm (10× NA0.45) for the redox pair NAD (P) H and FAD. Lifetime data is fitted for 2 component analysis for both NAD (P) H & FAD in SPCImage (B&H). Color coded FLIRR map is generated which shows heterogeneity in prostate tumor's redox states. The blue color regions have high FLIRR ratio whereas the tumor region shows lower FLIRR ratio in orange-yellow color.

FIGS. 5A-5B and 5D-5E. Comparison of intensity-based ratio vs FLIM ratio (FLIRR). Top panel intensity-based ratio and the bottom panel FLIRR. AA prostate cancer cells, pretreated overnight with 50 μM CoCl₂, imaged as control, Since Complex I is a major binding partner for NADH-a₂% fraction, the inhibition by CoCl₂ causes the NADH-a₂%/FAD-a₁% ratio to decrease and then increase upon stimulation with 25 mM glucose (A, D). Similar trends are observed upon starvation and stimulation with glucose in AA PCa (B, E) cells. The black arrow indicates increase in sensitivity in the FLIRR compared to the intensity ratio.

FIG. 6. Schematic of 2-photon FLIM microscope. LS-ti-sapphire IR pulsed laser; L-lens; PD-photodiode; BE-beam expander; M-mirrors; TL-tube lens; SL-scan lens; GM-Galvomirror; DR-dichroic mirror; OL-objective lens; EFW-emission filter wheel; FM-Flippable Mirrors; FM1—for inverted or upright configuration; RGB-Color filter for the H&E staining; S-Shutter; xyz-motorized stage.

FIG. 7. An embodiment of an AI/ML approach for cancer, such as PCa, grading and diagnosis.

FIG. 8. AE feature separates control vs doxorubicin treatment group in PCa cells.

FIGS. 9A-9B. Performance of ML feature in differentiation of treatment groups. [A] Kernel density histogram of ML feature. [B] Comparison of FLIRR and ML feature KS statistics, showing that the ML feature has a greater distribution shift from control.

### DETAILED DESCRIPTION

The following definitions are included to provide a clear and consistent understanding of the specification and claims. As used herein, the recited terms have the following meanings. All other terms and phrases used in this specification have their ordinary meanings as one of skill in the art would understand. Such ordinary meanings may be obtained by reference to technical dictionaries, such as Hawley's Condensed Chemical Dictionary 14th Edition, by R. J. Lewis, John Wiley & Sons, New York, N.Y., 2001.

References in the specification to “one embodiment,” “an embodiment,” etc., indicate that the embodiment described may include a particular aspect, feature, structure, moiety, or characteristic, but not every embodiment necessarily includes that aspect, feature, structure, moiety, or characteristic. Moreover, such phrases may, but do not necessarily, refer to the same embodiment referred to in other portions of the specification. Further, when a particular aspect, feature, structure, moiety, or characteristic is described in connection with an embodiment, it is within the knowledge of one skilled in the art to affect or connect such aspect, feature, structure, moiety, or characteristic with other embodiments, whether or not explicitly described.

The singular forms “a,” “an,” and “the” include plural reference unless the context clearly dictates otherwise. Thus, for example, a reference to “a compound” includes a plurality of such compounds, so that a compound X includes a plurality of compounds X. It is further noted that the claims may be drafted to exclude any optional element. As such, this statement is intended to serve as antecedent basis for the use of exclusive terminology, such as “solely,” “only,” and the like, in connection with any element described herein, and/or the recitation of claim elements or use of “negative” limitations.

The term “and/or” means any one of the items, any combination of the items, or all of the items with which this term is associated. The phrase “one or more” is readily understood by one of skill in the art, particularly when read in context of its usage. For example, one or more substituents on a phenyl ring refers to one to five, or one to four, for example if the phenyl ring is di-substituted.

As used herein, “or” should be understood to have the same meaning as “and/or” as defined above. For example, when separating a listing of items, “and/or” or “or” shall be interpreted as being inclusive, e.g., the inclusion of at least one, but also including more than one of a number of items, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e., “one or the other but not both”) when preceded by terms of exclusivity, such as “either,” “one of,” “only one of,” or “exactly one of.”

As used herein, the terms “including,” “includes,” “having,” “has,” “with,” or variants thereof, are intended to be inclusive similar to the term “comprising.”

The term “about” can refer to a variation of +5%, +10%, +20%, or +25% of the value specified. For example, “about 50” percent can in some embodiments carry a variation from 45 to 55 percent. For integer ranges, the term “about” can include one or two integers greater than and/or less than a recited integer at each end of the range. Unless indicated otherwise herein, the term “about” is intended to include values, e.g., weight percentages, proximate to the recited range that are equivalent in terms of the functionality of the individual ingredient, the composition, or the embodiment. The term about can also modify the endpoints of a recited range as discuss above in this paragraph.

Throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible sub-ranges as well as individual numerical values within that range. For example, description of a range such as from 1 to 6 should be considered to have specifically disclosed sub-ranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

In some aspects of the present invention, software executing the instructions provided herein may be stored on a non-transitory computer-readable medium, wherein the software performs some or all of the steps of the present invention when executed on a processor.

As will be understood by the skilled artisan, all numbers, including those expressing quantities of ingredients, properties such as molecular weight, reaction conditions, and so forth, are approximations and are understood as being optionally modified in all instances by the term “about.” These values can vary depending upon the desired properties sought to be obtained by those skilled in the art utilizing the teachings of the descriptions herein. It is also understood that such values inherently contain variability necessarily resulting from the standard deviations found in their respective testing measurements.

As will be understood by one skilled in the art, for any and all purposes, particularly in terms of providing a written description, all ranges recited herein also encompass any and all possible sub-ranges and combinations of sub-ranges thereof, as well as the individual values making up the range, particularly integer values. A recited range (e.g., weight percentages or carbon groups) includes each specific value, integer, decimal, or identity within the range. Any listed range can be easily recognized as sufficiently describing and enabling the same range being broken down into at least equal halves, thirds, quarters, fifths, or tenths. As a non-limiting example, each range discussed herein can be readily broken down into a lower third, middle third and upper third, etc. As will also be understood by one skilled in the art, all language such as “up to,” “at least,” “greater than,” “less than,” “more than,” “or more,” and the like, include the number recited and such terms refer to ranges that can be subsequently broken down into sub-ranges as discussed above. In the same manner, all ratios recited herein also include all sub-ratios falling within the broader ratio. Accordingly, specific values recited for radicals, substituents, and ranges, are for illustration only; they do not exclude other defined values or other values within defined ranges for radicals and substituents.

One skilled in the art will also readily recognize that where members are grouped together in a common manner, such as in a Markush group, the invention encompasses not only the entire group listed as a whole, but each member of the group individually and all possible subgroups of the main group.

Additionally, for all purposes, the invention encompasses not only the main group, but also the main group absent one or more of the group members. The invention therefore envisages the explicit exclusion of any one or more of members of a recited group. Accordingly, provisos may apply to any of the disclosed categories or embodiments whereby any one or more of the recited elements, species, or embodiments, may be excluded from such categories or embodiments, for example, for use in an explicit negative limitation.

The term “contacting” refers to the act of touching, making contact, or of bringing to immediate or close proximity, including at the cellular or molecular level, for example, to bring about a physiological reaction, a chemical reaction, or a physical change, e.g., in a solution, in a reaction mixture, in vitro, or in vivo.

The term “standard,” as used herein, refers to something used for comparison. For example, it can be a known standard agent or compound which is administered and used for comparing results when administering a test compound, or it can be a standard parameter or function which is measured to obtain a control value when measuring an effect of an agent or compound on a parameter or function. Standard can also refer to an “internal standard”, such as an agent or compound which is added at known amounts to a sample and is useful in determining such things as purification or recovery rates when a sample is processed or subjected to purification or extraction procedures before a marker of interest is measured. Internal standards are often a purified marker of interest which has been labeled, such as with a radioactive isotope, allowing it to be distinguished from an endogenous marker.

### Flim/Flirr

Fluorescence Lifetime Imaging Microscopy (FLIM) is a powerful tool to assess the metabolic state of cells and tissues under different pathophysiological conditions using the auto-fluorescent properties of metabolic co-enzymes NAD (P) H and FAD. Both are predominantly located in the mitochondrial tricarboxylic acid cycle (TCA) and electron transfer chain (ETC)-FAD exclusively-producing ATP. NAD (P) H is also present in the cytosolic glycolysis pathway, important for highly proliferating cells like cancer. The glycolysis pathway in the cytosol releases NADH and contributes to the free NADH pool. Mitochondrial oxidative phosphorylation (OXPHOS) activity consumes NADH (increased NADH-enzyme-bound fraction) and produces FAD (diminished FAD enzyme-bound fraction). Both the co-enzymes in their reduced (NAD (P) H and FADH₂) and oxidized (NAD (P)+ and FAD) forms participate in the cellular oxidation-reduction reactions critical for cell physiology. Since the fluorescence spectra of NADH and NADPH cannot be readily distinguished, NAD (P) H can used.

The fluorescence lifetimes of NAD (P) H and FAD are sensitive to changes in pH, temperature, their conformational state and proximity to quenchers. These co-enzymes exist in “free” or “enzyme-bound” states during cellular metabolic activity. FLIM is a very sensitive tool which allows discriminating the lifetimes and their relative fractions of free and enzyme-bound states of the co-enzymes from their fluorescence lifetime decay curve. Typically, fitting of lifetime decays of NAD (P) H and FAD are based on a two-component exponential decay model. The shorter (0.4 ns) and longer (2.4 ns) lifetimes of NAD (P) H represent the “free” and “enzyme-bound” components, respectively. On the other hand, the shorter (0.12 ns) and longer (3.38 ns) lifetimes of FAD represent the “enzyme-bound” and “free” components, respectively. As mentioned above, NAD (P) H auto-fluorescence signals come from the cytosol and mitochondria whereas, FAD signals mostly originate from the mitochondria thus, both are regarded as reporters of metabolic activity; their ratios are used as a marker of cellular redox states.

The enzyme-bound fractions of NAD (P) H (a₂%) and FAD (a₁%) and their lifetimes (tm) are used to quantify these metabolic changes, in particular with our novel NAD (P) H-a₂%/FAD-a₁% FLIM-based redox ratio. The commonly used intensity-based redox ratio [NAD (P) H/FAD], based predominantly on signals driven by mitochondrial OXPHOS is defined as a reduction of this ratio, due to the conversion of fluorescent NAD (P) H to non-fluorescent NAD+ and conversion of non-fluorescent FADH₂ to fluorescent FAD. The above FLIM-based NADH-a₂%/FAD-a₁% ratio can be used to avoid potential intensity-related artefacts (due to photo-bleaching, fluctuations in illumination sources, etc.) as a sensitive indicator of mitochondrial redox state. An increase in metabolic activity is defined as an increase of this ratio, due to the increase of the fluorescent NAD (P) H enzyme-bound lifetime fraction a₂% and decrease of the fluorescent FAD enzyme-bound fraction a₁%.

Auto-fluorescent biomarker, tryptophan (Trp), has also been linked to cancer investigations. Trp, an essential amino acid, is a precursor of niacin, which in turn is a precursor of NAD (P) H. Increased Trp catabolismand increased indoleamine 2,3-dioxygenase (IDO) activity in the kynurenine pathway are linked to cancer development and progression. Therefore, probing Trp is clinically relevant. In general, Trp fluorescence intensity and lifetime mainly provide information on the protein composition, protein structure of which they are a part of and changes in overall cellular microenvironment. Trp is therefore used as a marker for protein abundance. Like NAD (P) H and FAD, Trp also has shorter (0.5 ns-2.5 ns) and longer (3.1 ns) lifetimes which represent the “protein-bound” (as residues in proteins) and “free” (free amino acid) lifetime components, respectively. One of the major applications of FLIM is the measurement of Förster resonance energy transfer or FRET. In FLIM-FRET measurements, FRET events are identified if there is reduction in the donor lifetime, because of quenching of its fluorescence in the presence of the acceptor. Trp-NAD (P) H is a known FRET pair. The NAD (P) H-interacting enzymes carrying Trp residues from different metabolic pathways are potentially responsible for the quenching of Trp resulting in FRET. The efficiency of energy transfer or E % is calculated from the following equation E=1-τm/τ0, where τm is the mean lifetime of Trp in cells and τ0=3.1 ns of unquenched Trp lifetime measured from Trp in solution. Trp-NAD (P) H interactions can be used as a reporter of metabolic activity.

FLIM instrumentation can include a 3-channel FLIM imaging system of a Zeiss LSM 780 confocal/multiphoton (MP) laser scanning system coupled to the Zeiss inverted epi-fluorescence microscope, which is controlled with the ZEN software (Carl Zeiss, Inc). Multiphoton excitation of NAD (P) H and FAD was achieved by using an ultrafast (150 fs) tunable Ti: sapphire laser (680-1060 nm), operating at 80 MHz repetition rate (Chameleon Vision II, Coherent, Inc.). To excite the NAD (P) H and Trp we used 740 nm (NAD (P) H: 2-photon ex ET480/40 cm; Trp: 3-photon ex, HQ360/40 cm) and 890 nm for FAD (2-photon ex, 540/40 cm). The fluorescence decay per pixel was measured using 3-channel SPC-150 TCSPC board (Becker & Hickl, GmbH) where the SPCM software was used to acquire the FLIM data (v. 8.91). A Zeiss 40×1.3NA oil, (EC Plan-Neuofluar, UV transmission is 60% at 340 nm) objective lens was used to focus the light on the sample and collect the emission for 60 s. The average power at the specimen plane (7 mW) and the acquisition time was chosen to reduce any photodamage to the cells.

After simultaneous acquisition of FLIM images for Trp, NAD (P) H and FAD, the florescence lifetime images were fitted for 2-components using SPCImage software (v. 5.5, Becker & Hickl). Number of parameters was generated including photon images, τ1, τ2, a₁%, a₂%, and ₓ2 for each pixel of each channel. Since, NAD (P) H signal matches with the mitochondrial morphology, the mitochondrial Regions of Interest (ROI) s were thresholded by 2×2 pixels/ROI using the NAD (P) H photon image. The generated mitochondrial ROIs were used for further FLIM data analysis. See, for example, Alam et al. Scientific Reports. 7:10451:DOI: 10.1038/s41598-017-10856-3, 2017, which is incorporate herein in its entirety.

Multiphoton FLIM enhances both axial resolution and depth of penetration, thus enabling sub-micron sensitivity throughout 3D tumor spheroid models that are hundreds of microns in diameter and are embedded within ECM analogs that are millimeters in thickness.

The relative proportions of free and bound NAD (P) H or FAD can be used to infer whether glycolysis or oxidative phosphorylation are the metabolic pathways used by cancer cells to fuel various cellular processes and have been shown to correlate with conventional metabolic assays such as mass spectrometry and seahorse flux analysis. The ratio of the bound NAD (P) H to bound FAD, the Fluorescence Lifetime Imaging Redox Ratio (FLIRR), is an optical biomarker to determine a cell's primary metabolic pathway with higher FLIRR values often consistent with a shift towards oxidative phosphorylation and lower values often associated with a shift towards glycolysis.

### AI/ML

Machine learning (ML) is a branch of artificial intelligence (AI) that involves the use of computer systems that are able to learn and adapt without following explicit instructions, by focusing on data, algorithms and statistical models to analyze and draw inferences from patterns in data. In various embodiments, different methods of training the ML/AI model may be used. In one embodiment, the model can be trained with image batches (for example, 512 pixels×512 pixels×256 binsxn batches or 256 images (16×16) tiled at 128×128 pixel size). In this case, the model will directly generate a FLIM image (512 pixels×512 pixels) given the input with the dimension as (512 pixels×512 pixels×256 bins). Data from adjacent pixels or adjacent batches may be used to further improve the details of the FLIM image, achieving a super-resolution FLIM with high speed.

### Cancer/Treatment

Also provided herein is treatment of the cancer, such as treatment of solid tumors such as sarcomas and carcinomas (e.g., fibrosarcoma, myxosarcoma, liposarcoma, chondrosarcoma, osteogenic sarcoma, osteosarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, pancreatic cancer, pancreatic ductal adenocarcinoma, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilm's tumor, cervical cancer, uterine cancer, testicular cancer, lung carcinoma, small cell lung carcinoma, bladder carcinoma, epithelial carcinoma, glioma, astrocytoma, medulloblastoma, craniopharyngioma, ependymoma, pincaloma, hemangioblastoma, acoustic neuroma, oligodenroglioma, schwannoma, meningioma, melanoma, neuroblastoma, and retinoblastoma).

In some aspects, the subject/patient is treated with surgery, radiation, chemotherapy, immunotherapy and/or thermotherapy before, after or during the methods provided herein.

### EXAMPLES

The following examples are provided in order to demonstrate and further illustrate certain embodiments and aspects of the present invention and are not to be construed as limiting the scope thereof.

### Example I

Provided herein is a novel translational non-invasive method called FLIRR—of metabolic co-enzymes NAD (P) H & FAD-particularly to overcome the intensity-based light-scattering and absorption barriers in thicker specimens-2D vs 3D and in vivo imaging in animal models to understand cellular metabolism by FLIRR in cancer (Alam et al., 2017; incorporated in its entirety by reference). FLIRR can be used for understanding the role of redox state in tumor vs non-tumor, metastases & heterogeneity in drug response.

Of use herein are translational non-invasive small molecules for a screening technique called Trp-NAD (P) H FRET interaction. This approach will not lead to any cytotoxicity of the cancer or normal cells. As shown in the enclosed paper (Alam et al., 2017), this technique can quantitatively monitor the interactions under the apoptotic conditions.

Using the label free approach of FLIRR, it was demonstrated in PCa an increase in FLIRR of metabolic co-enzymes NAD (P) H & FAD and increase in Trp-NAD (P) H FRET interactions as early as 15 mins of anti-cancer drug-doxorubicin treatment in real time, much before the actual induction of apoptosis, validating these FLIRR parameters as early predictors of drug response in cancer (Alam et al., 2017).

For example, prostate cancer (PCa), a form of solid tumor is one of the leading cancers in men in the USA. Lack of experimental tools that predict therapy response is one of the limitations of current therapeutic regimens. In prostate cancer, reduced mtDNA and mitochondrial dysfunction has been demonstrated (Chandra et al., 2002). Also, reduced mtDNA and defective OXPHOS have been linked with the resistance and/or attenuation to apoptosis in cancer cells (Dey and Moraes, 2000; Chandra et al., 2002). Thus, correction of mitochondrial dysfunction and induction of apoptosis are unique, promising strategies in cancer treatment.

Using Trp-NAD (P) H FRET interactions (E %) vs FLIRR median correlation we were able to categorize PCa cells into “Pre-Apoptotic”, “Apoptotic or Responsive” and “Slow Responders or Resistant”, one of the most sought-after goals in cancer treatment and follow up (Alam et al., 2017).

This translational assay, based on FLIRR & Trp-NAD (P) H FRET interaction as early predictors of drug response—can further be applied for drug screening to identify new potent drugs specific to cancer. The methodology also identifies heterogeneity in drug response at single cell level, shortens the drug screening time to within an hour in real time, much before the actual cell death or induction of apoptosis used as an end point in traditional drug screening approaches. In addition to FLIRR, machine learning (Artificial intelligence (AI)) will be used as well for drug screening.

Fluorescence Lifetime Imaging Microscopy (FLIM) is a non-invasive label free approach, where the fluorescent properties of the endogenous fluorophores-Trp and redox pair NAD (P) H/FAD in their native environment are exploited which are sensitive to changes in their cellular microenvironment.

### Strategy:

1. Transform the FLIRR & Trp-NAD (P) H FRET interaction assay into an HTS format (96 well or higher) for drugs (for example, Doxorubicin) and small molecules screening in the PCa cancer cell line or any other cancer cell line of interest. This approach could be used to monitor the mitochondrial dysfunction and drug response of any disease including cancer, diabetes, Alzheimer, etc. This technique will allow one to screen a library of compounds.
(a) This can be achieved with a 2-photon FLIM microscope set up (or by building a miniaturized system) where one can use the FLIM mosaic or tiling function of the Zeiss 780 multiphoton microscope coupled with 3-channel SPC-150 TCSPC board (Becker & Hickl, GmbH) for Trp, NAD (P) H and FAD lifetime imaging. The number of data provided by the FLIM technique, the right value (two parameters) related to the drug response is picked to calculate the FLIRR.
(b) FLIRR will be used as an indicator of drug response-1st line of HTS screening-leading to Primary Hits. Depending on the sample size there can be multiple 1st lines of HTS screening-identifying several primary Hits.
(c) Primary Hits of the 1st line of screening will be further evaluated with FLIRR & Trp-NAD (P) H FRET interactions to identify the heterogeneity in drug response-leading to Secondary Hits.

2. Secondary Hits will be evaluated for cytotoxicity and heterogeneity in drug response using co-cultures of cancer and non-cancerous fibroblasts using the instant assay leading to—Tertiary Hits.

3. Tertiary Hits will further be evaluated by FLIRR and Trp-NAD (P) H FRET interactions in 3D models of cancer cell line derived spheroids which will require some optimizations moving from 2D to 3D culture.

4. Tertiary Hits will be evaluated by FLIRR and Trp-NAD (P) H FRET interactions in patient biopsy derived organoids-leading to the identification and development of personalized medicine approach.

5. A machine learning (AI) approach will also be used using more FLIM parameters to cross check the drug screening results by FLIRR.

6. Finally, looking into the mechanism of action of the “Lead compound” at the subcellular level by advanced fluorescence and/or super resolution microscopy.

### BIBLIOGRAPHY

1. Alam, S. R., Wallrabe, H., Svindrych, Z., Chaudhary, A. K., Christopher, K. G., Chandra, D. and Periasamy, A. (2017) Investigation of Mitochondrial Metabolic Response to Doxorubicin in Prostate Cancer Cells: An NADH, FAD and Tryptophan FLIM Assay. Sci. Rep. 7:10451. DOI: 10.1038/s41598-017-10856-3. www.nature.com/srp
2. Wallrabe, H., Svindrych, Z., Alam, S. R., Siller, K. H., Wang, T., Kashatus, D., Hu, S. and Periasamy, A. (2018). Segmented cell analyses to measure redox states of autofluorescent NAD (P) H, FAD & Trp in cancer cells by FLIM. Sci. Rep. 8:79. DOI: 10.1038/s41598-017-18634-x. www.nature.com/srp. Contributed equally.
3. Rehman, S., O'Melia, M. J., Wallrabe, H., Chandra, D., Svindrych, Z. and Periasamy, A. (2018) Multiphoton FLIM-FRET microscopy to investigate the NAD (P) H and tryptophan interactions in cells and tissues. In “Modern Laser microscopy and fluorescence lifetime imaging” Ed. Karsten Koenig, DeGruyter Press, Berlin. Ch. 7, pp 141-162.
4. Periasamy, A., Alam, S. R., Svidrych, Z. and Wallrabe, H. (2017) FLIM-FRET Image Analysis of Tryptophan in Prostate Cancer Cells, Proc SPIE (ECBO) 10414:1041402-Pp1-5.
5. Alam, S. R., Wallrabe, H., Svindrych, Z., Chandra, D. and Periasamy, A. (2017) Effects of Anti-Cancer Drug Doxorubicin on Endogenous Biomarkers NAD (P) H, FAD & Trp in prostate cancer cells-a FLIM Study. Proc. of SPIE, Vol. 10069: 100691L-Pp1-6.
6. Rehman, S., O'Melia, M. J., Wallrabe, H., Svindrych, Z. and Periasamy, A. (2016) Investigation of prostate cancer cells using NADH and Tryptophan as biomarker: multiphoton FLIM-FRET microscopy. Proc. SPIE Int. Soc. Opt. Eng.7712: 97120Q. pp1-5.

### Example II-A Novel FLIRR Assay for Biopsy Tissues Screening and Grading

Prostate cancer (PCa) is a leading cause of cancer and the second leading cause of death in men in the USA (CDC, USA). Alarmingly, the incidence of PCa has been on the rise and 1 in 7 men will be diagnosed with PCa in their lifetime. Increasingly, men with PCa are diagnosed late at advanced stages, where 35% of the cases have recurrence leading to transformation into a castration resistant prostate cancer (CRPC) phenotype, the most deadly and aggressive form of PCa where standard chemotherapies are largely ineffective. Early diagnosis, where chemotherapeutics is most effective will help ameliorate the burden of PCa.

Routine screening of PCa involves a PSA (Prostate Specific Antigen) test and a digital rectal exam (DRE). Normal PSA level is under 4 nanograms per milliliter (ng/ml) of blood while 10 and above suggests a high risk of cancer, with few exceptions: (1) men can have prostate cancer with a PSA less than 4; (2) a prostate that is inflamed (prostatitis) or enlarged can boost PSA levels, yet further testing may show no evidence of cancer. If the PSA test is abnormal a biopsy is required to detect cancer. Biopsy and Gleason Grading is based on histopathology. The grading system is entirely based on the arrangement of abnormal carcinoma cells in the Hematoxylin and Eosin (H&E) stained biopsied tissue (Humphrey 2004). The Gleason score isn't a separate test. It's a number based on the results of the biopsy. The pathologist grades 1 to 5 to the most common (primary) and second most common (secondary) patterns of cells found in a tissue sample: The two grades added together is the tissue's Gleason score. Cancers typically score 6 or more. A score of 7 means the cancer is intermediate, and a higher score (8 to 10) means the cancer is more likely to grow and spread. Gleason score combined with the results of the PSA blood test and digital rectal exam are used to assess how advanced the prostate cancer may be. It is important to note the cancer grading is done by pathologists looking at the biopsy tissue under the microscope to identify the normal and cancerous cells.

On the other hand, it is provided herein a novel automatic screening of biopsy tissue to identify and grade the cancer progression without any labeling.

In cancer there is re-programming of cellular metabolism. Mitochondrial oxidative phosphorylation (OXPHOS) is largely defective and cellular energy demands are met through hyperactive glycolytic pathway, referred to as the “Warburg Effect. This characteristic shift in cancer cell metabolism has been linked with cell proliferation, progression, and metastasis. Fluorescence Lifetime Imaging Microscopy (FLIM) of endogenous, auto-fluorescent metabolic co-enzymes NAD (P) H and FAD is a powerful non-invasive, label-free approach to assess the metabolic alterations in cancer. Since, the metabolic co-enzyme are acceptable markers of cellular redox state we have developed a novel Fluorescence Lifetime based Redox Ratio (FLIRR) (Alam et al., 2017 & 2022; Wallrabe et al., 2018; Cao et al., 2020) for identifying cancer vs non-cancer and assessing cancer heterogeneity in the biopsied tissue.

Routine histopathology is done with H&E staining. FLIRR has many advantages over traditional H&E staining. FLIRR is a dye free/label free approach which does not involve any labeling, acid-alcohol differentiation, dehydration and washing steps required for H&E staining, thereby preventing any cross-contamination and false positivity across different biopsied tissue sections being processed together. FLIRR exploits the lifetimes of endogenous, auto-fluorescent fluorophores NAD (P) H and FAD, the redox pairs-which are sensitive to changes in their microenvironment. Hence, FLIRR will discriminate normal versus tumor microenvironment based on their redox status and helps to detect the cancer at earlier stage. Moreover, FLIRR detects cancer cells at the molecular level compared to H&E staining. Since neoplastic transformation in prostate epithelium takes longer for H&E than the molecular level detection of cancer using FLIRR.

SPAD camera or image intensifier-based gating camera or any PMT based FLIM detectors could be used for screening of the whole tissue like plate scanner (or reader) for screening the biopsy tissue.

The high-speed FLIM detector or a SPAD array camera to monitor the NAD (P) H and FAD auto-fluorescent signal from the tissue after IR excitation with 800 nm or 780 nm or two wavelengths excitation (NADH 740 nm and FAD 890 nm) Pico or femtosecond pulsed IR laser, then capturing the emission signals for NAD (P) H 450/50 nm and FAD 520/40 nm. We will have the xyz stage for automatic screening of the biopsy tissue and a wave front sensor to improve the signal at various depths of the tissue.

The FLIRR value could be automatically calculated at each pixel of the image. This calculation may be using time corelated single photon counting or fast FLIM frequency domain. The FLIM analysis may be implemented by existing maximum likelihood (MLE) fitting methods or phasor analysis. A large volume of data points generated will be further used by the AI/machine learning module to speed-up the data display. The screening speed will not be affected by the data analysis since each pixel acquisition will immediately be processed to calculate the FLIRR value. The accumulated FLIRR value will be then used for the AI system for evaluation of the cancer.

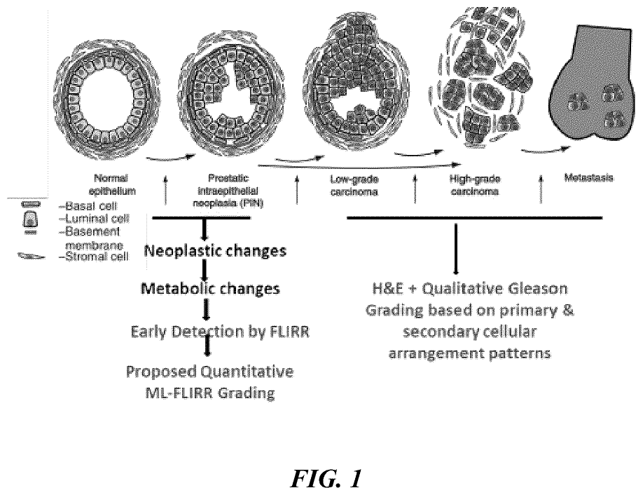

FLIRR is the most accurate way to screen the biopsy tissue as shown in FIGS. 1-3). A less accurate way one can also follow only the bound NAD (P) H to compare the signal with H&E staining.

The novel FLIRR system with AI analysis (Mbogo et al., 2023) is a sensitive, unbiased approach in tumor evaluation and grading. This will aid in the early diagnosis of tumor which greatly increases the chances of a successful treatment. An innovative approach like this offers precision in cancer screening and diagnosis not only for PCa but for other cancers as well.

### BIBLIOGRAPHY

1. Alam, S. R. Wallrabe, H., Svindrych, Z., Chaudhary, A. K., Christopher, K. G., Chandra, D. and Periasamy, A. (2017) Investigation of Mitochondrial Metabolic Response to Doxorubicin in Prostate Cancer Cells: An NADH, FAD and Tryptophan FLIM Assay. Sci. Rep. 7:10451. DOI: 10.1038/s41598-017-10856-3. www.nature.com/srp
2. Wallrabe, H., Svindrych, Z., Alam, S. R., Siller, K. H., Wang, T., Kashatus, D., Hu, S. and Periasamy, A. (2018) Segmented cell analyses to measure redox states of autofluorescent NAD (P) H, FAD & Trp in cancer cells by FLIM. Sci. Rep. 8:79. DOI: 10.1038/s41598-017-18634-x. www.nature.com/srp.
3. Cao, R., Wallrabe, H. and Periasamy, A. (2020) Multiphoton FLIM imaging of NAD (P) H and FAD with one excitation wavelength. J. Biomed. Opt. 25 (1), 014510. doi: 10.1117/1.JBO.25.1.014510
4. Alam, S. R., Wallrabe, H., Christopher, K. G., Siller, K., and Periasamy, A. (2022) Characterization of mitochondrial dysfunction due to laser damage by 2-photon FLIM microscopy. Scientific Reports. Scientific Reports 12, 11938 (2022) (Nature journal group).
5. Mbogo, B. P., Siller, K. H., Zhang, J., Wallrabe, H., Alam, S. R., Periasamy, A. (2023) Optimizing Machine Learning Hyperparameters in Two-photon FLIM Image Analysis. Proc. SPIE 12384, Multiphoton Microscopy in the Biomedical Sciences XXII, Proc. SPIE doi: 6.1117/12.2666216 https://www.spicdigitallibrary.org/terms-of-use.

### Example III

Metabolic Imaging: 2p-FLIRR Microscopy with SPAD Camera

Metabolic reprogramming, defined as the shift from oxidative phosphorylation (OXPHOS) to glycolysis has been long identified as a common factor in the etiology of cancer. Cancer cells to support their bioenergetic and biosynthetic demands during tumorigenesis shift gears to the glycolytic pathway vs OXPHOS, described as the Warburg Effect (12). Moreover, this characteristic shift in cancer cells' metabolism takes place much earlier in the disease process which can be delineated by imaging of the metabolic coenzymes NAD (P) H and FAD (13,14).

Auto-fluorescent signals of the coenzymes NAD (P) H and FAD is commonly tracked by an intensity-based redox ratio to measure the overall redox state in cells because FAD/NAD (P) H are usually in near oxidation-reduction equilibrium. Therefore, the ratio of the two fluorescence intensities, suitably normalized, approximates the in vivo oxidation-reduction which offers a foundation for the resolution of the redox states in 2- and 3-D models. Unfortunately, optical artefacts in light illumination, such as scattering in biological specimens and differential absorption at various depths in tissues-makes intensity-based methods less sensitive. As an alternative, provided herein is a non-invasive, highly sensitive FLIM-based method (FLIRR) to monitor glycolysis vs OXPHOS to stack microenvironmental changes in translational research. Other FLIM based metrics have also been used to understand metabolic alterations in cancer. Despite the encouraging progress made using traditional slow PMT (Photomultiplier tube) based metabolic imaging, the high-speed high-sensitivity SPAD camera can provide dynamic imaging of NAD (P) H and FAD using single wavelength excitation (800 nm or 780 nm). The integration of the SPAD camera with FLIM (e.g., 2p-FLIM) system advances the screening of biopsied cancer/tumors, such as PCa tissue. These techniques can provide absolute quantification of the redox ratio (FLIRR) in biopsied tissue.

### Prostate Cancer Diagnosis in Heterogeneous Microenvironment

Routine screening of PCa involves a blood PSA (Prostate Specific Antigen) test and a digital rectal exam (DRE). Normal PSA level is under 4 nanograms per milliliter (ng/ml) of blood while 10 and above suggests a high risk of cancer, with few exceptions: (1) men can have prostate cancer with a PSA less than 4; (2) a prostate that is inflamed (prostatitis) or enlarged can boost PSA levels, yet further testing may show no evidence of cancer. If the PSA test is abnormal a biopsy is required to detect cancer. The gold standard Gleason grading of the biopsy yields important prognostic information which is the basis for treatment regime of the PCa patients. Based on the H&E staining of the biopsied tissue, the pathologist characterizes the tumor in different Gleason growth patterns based on the histological architecture of the tumor tissue. Gleason grading is based on the arrangement of the primary and secondary patterns of the cells in the tissue according to the guidelines from the International Society of Urological Pathology (ISUP). Gleason grading however, is subject to high inter-observer variability leading to over- or under-treatment of PCa patients and recurrence of PCa affecting their health, and daily life.

The approach provided herein is an innovative, sensitive diagnostic tool. The tumor microenvironment is structurally and functionally heterogeneous which leads to the spatial heterogeneity in metabolic reprogramming. The FLIRR (e.g., 2p-FLIRR) microscopy technique is sensitive to resolve this heterogeneity in cells and tissues.

### Machine Learning (ML) Based FLIRR Vs Gleason Grading.

Drug resistance is the primary cause of failure of the chemotherapies in most cancers. Growing evidence suggests that metabolic reprogramming in cancer potentially contributes to the heterogeneity in treatment outcomes and drug resistance.

FLIRR has many advantages over traditional H&E staining for routine histopathology and Gleason scoring of biopsies. FLIRR is a dye free/label free approach which does not involve any fixation, labeling, acid-alcohol differentiation, dehydration and washing steps required for H&E staining, thereby preventing any cross-contamination and false positivity across different biopsied tissue sections being processed together. FLIRR will delineate the changes in redox states of tumor and non-tumor by fluorescence lifetime parameters of NAD (P) H and FAD, the redox pairs being sensitive to changes in their tumor microenvironment. Hence, FLIRR (e.g., 2p-FLIRR) approach to demarcate the differences in the redox states in tumor lesions vs normal proliferating cells in the prostate tissues is a robust and sensitive diagnostic index for detecting cancer at the earliest stages (FIG. 1).

Machine learning (ML) technology is getting more accessible in cancer diagnosis. The ML involves several different techniques able to process large amounts of data and reduce data dimensionality. Our experience in the development of ML approach for monitoring drug response in PCa cells, indicates that the development of ML is required to handle the large volume of data generated by the FLIRR assay of the biopsied tissue. The data set can be augmented by running simulations. The ranges of FLIRR scores can be used by the AE (Autoencoder) trained model or other suitable ML models like long-short term memory (LSTM), autoregressive models, or time delay neural networks for evaluation and tumor grading. The novel FLIRR assay is a robust, sensitive, unbiased approach in tumor evaluation and grading. The innovative FLIRR approach offers precision in cancer screening and diagnosis not only for PCa but for other cancers as well where metabolic reprogramming plays a role.

2p-FLIRR Assay: A Promising Diagnostic Index

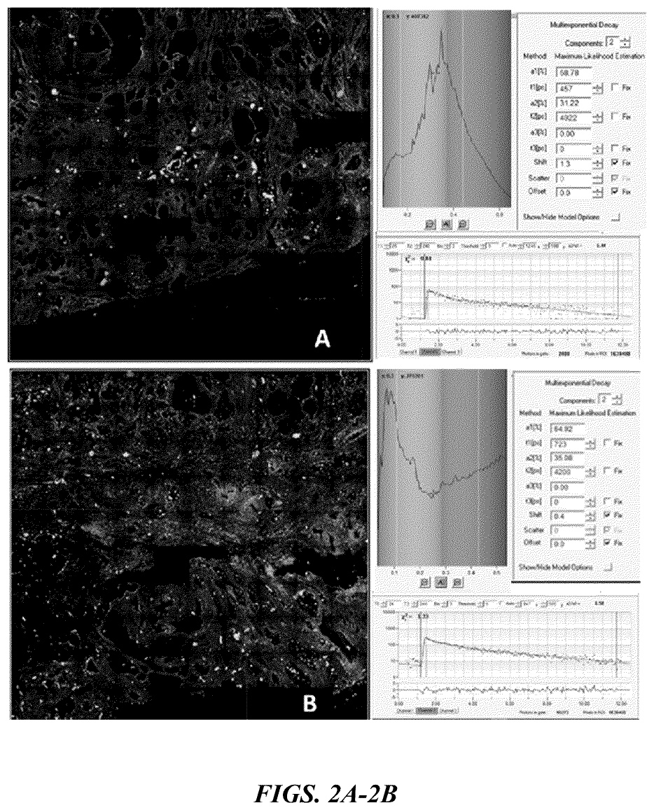

The assay is based on the novel FLIRR score as a diagnostic index. As a proof-of-principle we have performed 2-photon FLIM mosaic on PCa biopsied tissue and computed a FLIRR analysis (FIGS. 2 & 4). The FLIRR map, a color-coded image of the biopsied tissue section showed lower FLIRR scores for the tumor region (as expected due to defective OXPHOS) and surprisingly some regions in the control as well, which was diagnosed as “non-cancerous/normal” by H&E staining and Gleason grading. Thus, again emphasizing on the importance of FLIRR as a diagnostic index over H&E based Gleason scoring for early detection. This patient (FIG. 2) had a blood PSA level of 9.21 which is borderline of PCa biopsy requirement. This is one such example of detecting precancerous lesion by FLIRR early. Interestingly, the distribution of the FLIRR values of the precancerous lesions (FIG. 2A) were in between the 2 distribution peaks seen in the confirmed tumor, distribution of shorter FLIRR denoting tumor and longer FLIRR denoting normal regions (FIG. 2B).

### Development of ML Based FLIRR Grading: Sensitive Tool for Early Cancer Detection

Artificial Intelligence (AI) has shown promise in diagnosing prostate cancer in biopsies. Most of these AI methods are based on H&E and Gleason grading which is inherently subjective to high inter- and intra-pathologist variability or on virtual H&E images created by deep UV hyperspectral imaging which is photo-damaging and done on fixed specimen. Also, H&E identifies morphological changes which are late-onset changes, in primary and secondary patterns after the tumor has already progressed. Instead, herein we use non-photodamaging 2-photon FLIRR imaging on unfixed flash frozen biopsies, which are sensitive not only in diagnosing aggressive advanced tumor, but in the early detection and predicting the risk of developing prostate cancer based on the early on-set changes in the redox state during the early phase of neoplastic transformations and metabolic re-programming (FIG. 1, adapted and modified from Knudsen & Vasioukhin11). A quantitative ML based FLIRR grading method on the time scale of tumor progression (FIG. 1) is more sensitive and an unbiased approach for diagnosing PCa over AI based on qualitative H&E Gleason grading. Early diagnosis of tumor by FLIRR grading positively impacts the prognosis and increase the chance of successful treatment. FLIRR as a predictive biomarker for cancer diagnosis, resulting in better stratification or grading of the patients' biopsies based on quantitative FLIRR score.

FLIRR provides superior sensitivity for early detection of cancer therefore better prognosis and treatment. Furthermore, the addition of the AI/ML module will automate large volume of data processing, analysis, and unbiased grading of cancer, including PCa, based on

### Flirr Score.

A Sensitive 2p-FLIRR Assay with SPAD Camera for the Diagnosis of PCa in Biopsied Tissue.

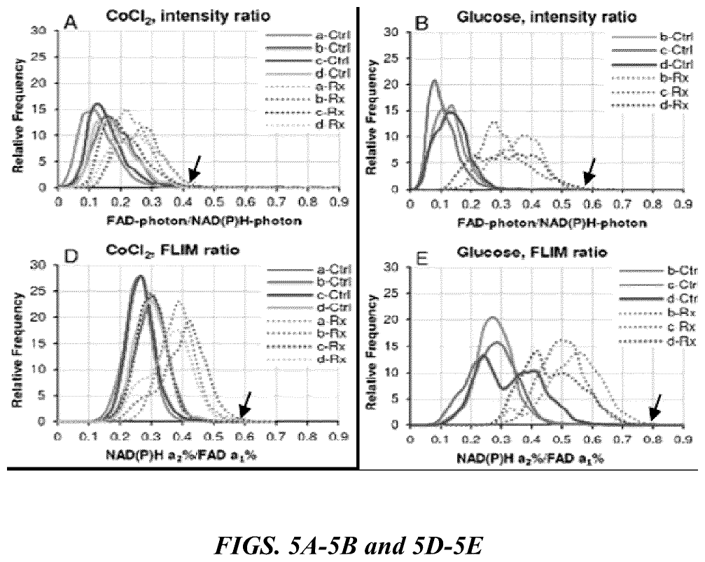

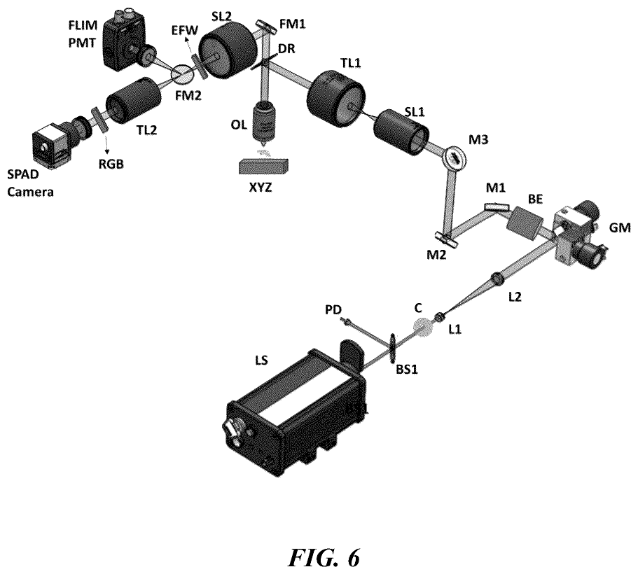

One embodiment provides a novel and an innovative FLIRR approach for assessing heterogeneity in treatment response in cancer, such as PCa, patient derived cells using a FLIM PMT based 2-photon system. As shown in FIG. 5, FLIRR provides improved sensitivity in response to different treatments and removes optical artefacts involved in optical imaging. The PMT based system, however, is slow in data collection. As shown in FIG. 6, one can integrate the high-speed sensitive Single Photon Avalanche Diode (SPAD) camera for FLIM with the existing PMT based (B & H) two-photon FLIM system (FIG. 6). In parallel, one can use the SPAD camera to acquire intensity-based H&E-stained images. This system can be configured as an inverted or upright microscope using the flip mirror FM1 (inverted configuration is not shown in FIG. 6). The 800 nm pulsed IR laser from Coherent Mira 900 will be scanned by the XY scanner (GM). Two pairs of telescopes (SL & TL) and beam expander (BE) are located in such a way as to adjust the excitation beam diameter to fill the back focal plane of the objective lens. The emission filter wheel is integrated before a flip mirror (FM2), which will be used for NAD (P) H (425-475 nm) and FAD (520-560 nm) to send the fluorescence signal to the FLIM PMT (Becker & Hikel) or to the SPAD camera (SPAD512S; 512×512 pixels; 400 fps for 8-bit; minimum gate shift 17 ps; zero readout noise). FLIM data from SPAD camera will be processed with the existing FLIM analysis for gating camera. FLIM PMT will be used as a reference to follow the SPAD camera acquired FLIM data. The high-speed time gated SPAD camera, with 390 frames per see at 8-bit, aids in the collection of large number of photons in less acquisition time for lifetime measurements.

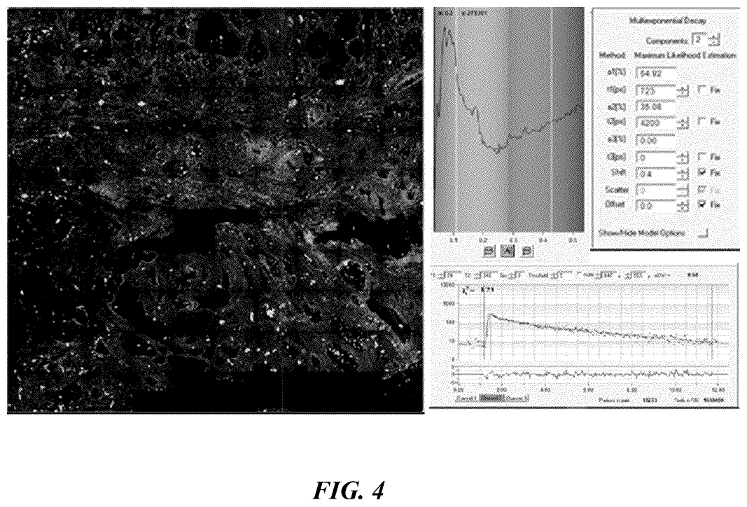

FLIRR is a sensitive assay based on the tumor's redox status for use on patient-derived biopsies, for diagnosis and grading of the tumor. FLIRR provides a grading method like the Gleason score for diagnosing and staging the cancer progression. For PCa screening, core needle biopsies under the guided transrectal ultrasound (TRUS) are performed with a typical core needle dimension of ½″×⅙″ (1.27 cm x0.158 cm). The biopsies are then further processed by formalin fixation, paraffin embedding, sectioning, and staining by contrasting reagents-H&E for routine histopathology. FLIRR assay can be performed on the unstained flash frozen tissue biopsies embedded in the optimal cutting temperature (OCT) compound (an inert material) on sections ˜10 μm thickness (1-2 cm in size). As the biopsied tissue is large (1-2 cm in size), the mosaic tiling images can be acquired using the motorized xyz stage of the microscope (FIG. 6) for the entire biopsy tissue sections using single wavelength excitation (800 nm) and simultaneously collecting the signals from the redox pairs NAD (P) H and FAD. FLIRR map (FIG. 4): a color-coded image of tumor's redox states will be generated and compared to the FLIRR map of the biopsied non-cancerous area of the tissue. 2p-FLIRR map enables one to separate tumor vs normal area and identify the heterogeneity in the redox states within the intact tumor sections. The findings of 2p-FLIRR were correlated with H&E histopathological examination and conventional Gleason scoring for PCa diagnoses, to devise a FLIRR based ML model for grading and diagnosis.

In PCa defective mitochondrial OXPHOS is reported. Therefore, a FLIRR=1 would be an ideal value where OXPHOS would be functioning normally. But since mitochondrial OXPHOS is a dynamic process with different mitochondria in tissue having differential

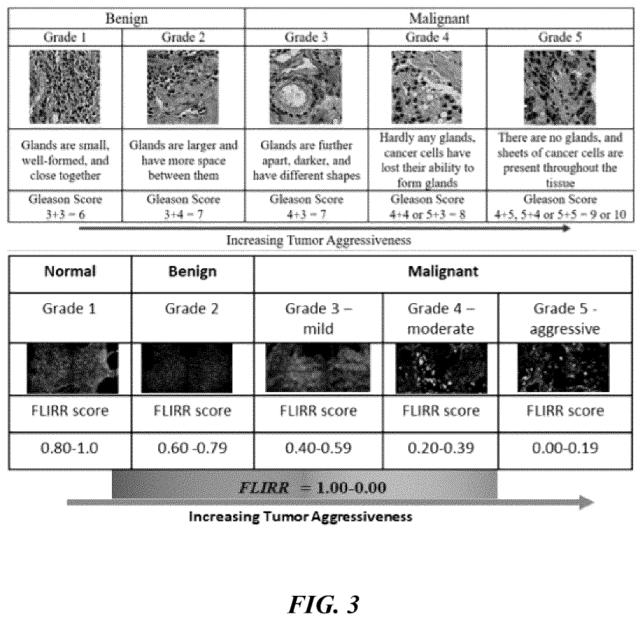

OXPHOS activity, a range of FLIRR values can be expected. In one embodiment: FLIRR ≥0.8-1.0 could be considered normal; FLIRR ≥0.60-0.79 benign, mild≤0.40-0.59; moderate≤ 0.20-0.39 and aggressive≤0.00-0.19. The findings were correlated with H&E staining and Gleason scoring to measure the robustness, sensitivity of the 2p-FLIRR assay for PCa tumor diagnosis and grading as shown in FIG. 3.

### Development and Validation of AI-Machine Learning Algorithms for PCa Prediction by FLIRR Assay.

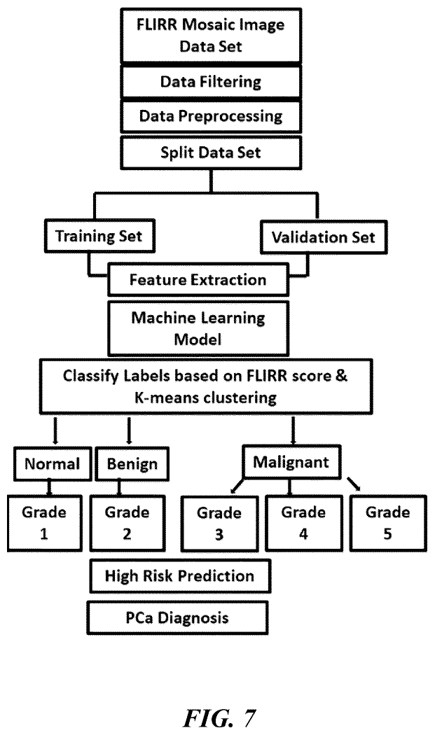

Analysis of the FLIRR mosaic data from, for example, 100 biopsies, each with a FLIM mosaic (256 images tiled at 16×16) in duplicates (x2, at least) from non-cancerous normal regions and tumor lesions will result in a large volume of data. These data can be further analyzed by AI/ML approaches. An unsupervised machine learning (ML) model for a grading method based on FLIRR scores and K-means clustering can be developed for diagnosing and grading the stage of the cancer, such as PCa. AI/ML trained model on FLIRR scores, would also be able to predict the risk of developing cancer, such as PCa. An AI/ML model for cancer, such as PCa, grading and diagnosis is presented in FIG. 7. However, the suitability of other ML models like long-short term memory (LSTM), autoregressive models, or time delay neural networks for their robustness can also be used. The results of AI/ML based grading can be compared with Gleason scoring to assess the sensitivity and specificity of the model in PCa diagnosis.

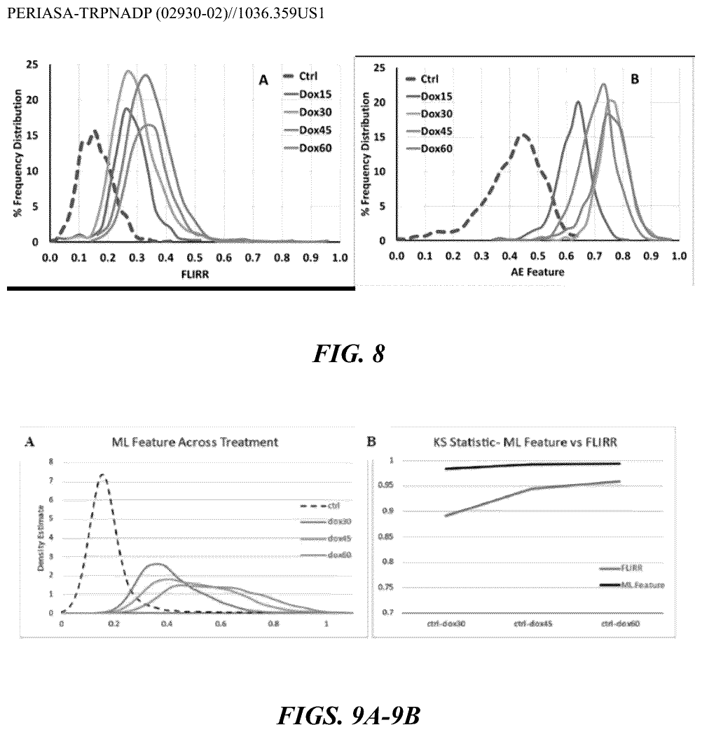

AI/ML can be used with an autoencoder (AE) an Artificial Neural Network (ANN) to learn efficient data representation by unsupervised learning. We have trained the AE model to analyze huge volume of FLIM data, for reduction of data dimensionality and ML Feature extraction. The AE feature extraction showed better performance in comparison to FLIRR in detecting differences in doxorubicin treatment groups temporally in patient derived PCa cells (FIG. 8). Also, the robustness of the AE model was further refined by tuning the hyperparameters (FIG. 9).

All publications, patents, and patent applications, Genbank sequences, websites and other published materials referred to throughout the disclosure herein are herein incorporated by reference to the same extent as if each individual publication, patent, or patent application, Genbank sequences, websites and other published materials was specifically and individually indicated to be incorporated by reference. In the event that the definition of a term incorporated by reference conflicts with a term defined herein, this specification shall control.

What is claimed is:

1. A method to screen for cancer treatment compounds comprising
contacting a cancer cell with a test compound and
determining the ratio of bound NAD (P) H to bound FAD (the fluorescence lifetime imaging redox ratio (FLIRR)) via fluorescence lifetime imaging microscopy (FLIM),
wherein an increased ratio of NAD (P) H to FAD, as compared to a cancer cell that has not been contacted with the test compound, indicates a cancer treatment compound.

2. The method of claim 1, wherein the compounds are further tested for cytotoxicity and heterogeneity in drug response using co-cultures of the cancer and non-cancerous cells.

3. The method of claim 2, wherein the cells are fibroblasts.

4. The method of claim 2, further comprising determining Trp-NAD (P) H FRET interactions in the cancer cells.

5. The method of claim 4, wherein the Trp-NAD (P) H FRET interactions are determined in a 3D model spheroid of cancer cells.

6. The method of claim 1, wherein the FLIM is multiphoton FLIM.

7. The method of claim 6, wherein photomultiplier tube (PMT) based metabolic imaging or SPAD camera with FLIM is used.

8. The method of claim 1, wherein FLIRR is calculated at each pixel of the image.

9. The method of claim 8, wherein the calculation uses time correlated single photon counting or fast FLIM frequency domain.

10. The method of claim 8, wherein the FLIM analysis is carried out by maximum likelihood estimation (MLE) fitting methods or phasor analysis.

11. A method to diagnose cancer a tissue biopsy comprising
determining the ratio of bound NAD (P) H to bound FAD (the fluorescence lifetime imaging redox ratio (FLIRR)) via fluorescence lifetime imaging microscopy (FLIM) in said biopsy,
wherein decreased ratio of NAD (P) H to FAD, as compared to a non-cancerous biopsy indicates a cancer.

12. The method of claim 11, wherein the FLIM is multiphoton FLIM.

13. The method of claim 12, wherein photomultiplier tube (PMT) based metabolic imaging or SPAD camera with FLIM is used.

14. The method of claim 11, wherein FLIRR is calculated at each pixel of the image.

15. The method of claim 14, wherein the calculation uses time correlated single photon counting or fast FLIM frequency domain.

16. The method of claim 14, wherein the FLIM analysis is carried out by maximum likelihood (MLE) fitting methods or phasor analysis.

17. The method of claim 11, wherein the biopsy is fresh or frozen and/or thawed.

18. The method of claim 11, wherein the cancer is a solid tumor such as a sarcoma or carcinoma.

19. The method of claim 18, wherein the cancer is a fibrosarcoma, myxosarcoma, liposarcoma, chondrosarcoma, osteogenic sarcoma, osteosarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, pancreatic cancer, pancreatic ductal adenocarcinoma, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilm's tumor, cervical cancer, uterine cancer, testicular cancer, lung carcinoma, small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodenroglioma, schwannoma, meningioma, melanoma, neuroblastoma, and retinoblastoma.

20. The method of claim 18, wherein the cancer is prostate cancer.

21. The method of claim 11, wherein no exogenous label or dye is used.

22. The method of claim 14, wherein the data is used by artificial intelligence (AI) to generate a data display and/or for cancer evaluation.

23. The method of claim 22, further comprising grading the cancer.

24. The method of claim 11, further comprising treating the diagnosed cancer.
